(19) United States
(12) Patent Application Publication
(10) Pub. No.: US 2024/0016560 A1
(43) Pub. Date: Jan. 18, 2024

(54) ROBOTIC DRIVE SYSTEM FOR A CATHETER-BASED PROCEDURE SYSTEM

(71) Applicant: Corindus, Inc., Newton, MA (US)

(72) Inventors: Cameron Canale, Groton, MA (US); Kody Saeedi, Ashland, MA (US); Saeed Sokhanvar, Belmont, MA (US); Chi Min Seow, Watertown, MA (US); Andrew Clark, Arlington, MA (US); Omid Saber, Waltham, MA (US); Eric Klem, Lexington, MA (US); Steven J. Blacker, Framingham, MA (US)

(21) Appl. No.: 18/255,375

(22) PCT Filed: Jan. 14, 2021
(86) PCT No.: PCT/US2021/070038

§ 371 Date: Jun. 1, 2023

(51) Int. Cl. A61B 34/37; A61B 17/12; A61G 13/10

(52) U.S. Cl. CPC A61B 34/37; A61B 17/12022; A61G 13/101; A61B 2034/301

(57) ABSTRACT

A robotic drive system for a catheter-based procedure system includes a positioning system coupled to a patient table, the patient table having a front side and a rear side. The rear side of the patient table has a rail. The robotic drive system further includes a linear member coupled to the positioning system at a connection point and at least three device modules coupled to the linear member. Each device module is independently controllable and includes a drive module having a front side and a cassette mounted on the drive module. The cassette has a front side and is configured to support an elongated medical device having a longitudinal device axis. The cassette is mounted on the drive module in a vertical orientation so that the front side of the cassette is parallel to the front side of the drive. In addition, a width defined between the longitudinal device axis of the elongated medical device and the connection point of the linear member to the positioning system is equal to or less than a distance between an insertion point for the elongated medical device to a patient and the rail on the rear side of the patient table.

### FIELD

The present invention relates generally to the field of robotic medical procedure systems and, in particular, to a robotic drive system for robotically controlling the movement and operation of elongated medical devices in interventional procedures.

### BACKGROUND

Catheters and other elongated medical devices (EMDs) may be used for minimally invasive medical procedures for the diagnosis and treatment of diseases of various vascular systems, including neurovascular intervention (NVI) also known as neurointerventional surgery, percutaneous coronary intervention (PCI) and peripheral vascular intervention (PVI). These procedures typically involve navigating a guidewire through the vasculature, and via the guidewire advancing a catheter to deliver therapy. The catheterization procedure starts by gaining access into the appropriate vessel, such as an artery or vein, with an introducer sheath using standard percutaneous techniques. Through the introducer sheath, a sheath or guide catheter is then advanced over a diagnostic guidewire to a primary location such as an internal carotid artery for NVI, a coronary ostium for PCI, or a superficial femoral artery for PVI. A guidewire suitable for the vasculature is then navigated through the sheath or guide catheter to a target location in the vasculature. In certain situations, such as in tortuous anatomy, a support catheter or microcatheter is inserted over the guidewire to assist in navigating the guidewire. The physician or operator may use an imaging system (e.g., fluoroscope) to obtain a cine with a contrast injection and select a fixed frame for use as a roadmap to navigate the guidewire or catheter to the target location, for example, a lesion. Contrast-enhanced images are also obtained while the physician delivers the guidewire or catheter so that the physician can verify that the device is moving along the correct path to the target location. While observing the anatomy using fluoroscopy, the physician manipulates the proximal end of the guidewire or catheter to direct the distal tip into the appropriate vessels toward the lesion or target anatomical location and avoid advancing into side branches.

Robotic catheter-based procedure systems have been developed that may be used to aid a physician in performing catheterization procedures such as, for example, NVI, PCI and PVI. Examples of NVI procedures include coil embolization of aneurysms, liquid embolization of arteriovenous malformations and mechanical thrombectomy of large vessel occlusions in the setting of acute ischemic stroke. In an NVI procedure, the physician uses a robotic system to gain target lesion access by controlling the manipulation of a neurovascular guidewire and microcatheter to deliver the therapy to restore normal blood flow. Target access is enabled by the sheath or guide catheter but may also require an intermediate catheter for more distal territory or to provide adequate support for the microcatheter and guidewire. The distal tip of a guidewire is navigated into, or past, the lesion depending on the type of lesion and treatment. For treating aneurysms, the microcatheter is advanced into the lesion and the guidewire is removed and several embolization coils are deployed into the aneurysm through the microcatheter and used to block blood flow into the aneurysm. For treating arteriovenous malformations, a liquid embolic is injected into the malformation via a microcatheter. Mechanical thrombectomy to treat vessel occlusions can be achieved either through aspiration and/or use of a stent retriever. Depending on the location of the clot, aspiration is either done through an aspiration catheter, or through a microcatheter for smaller arteries. Once the aspiration catheter is at the lesion, negative pressure is applied to remove the clot through the catheter. Alternatively, the clot can be removed by deploying a stent retriever through the microcatheter. Once the clot has integrated into the stent retriever, the clot is retrieved by retracting the stent retriever and microcatheter (or intermediate catheter) into the guide catheter.

In PCI, the physician uses a robotic system to gain lesion access by manipulating a coronary guidewire to deliver the therapy and restore normal blood flow. The access is enabled by seating a guide catheter in a coronary ostium. The distal tip of the guidewire is navigated past the lesion and, for complex anatomies, a microcatheter may be used to provide adequate support for the guidewire. The blood flow is restored by delivering and deploying a stent or balloon at the lesion. The lesion may need preparation prior to stenting, by either delivering a balloon for pre-dilation of the lesion, or by performing atherectomy using, for example, a laser or rotational atherectomy catheter and a balloon over the guidewire. Diagnostic imaging and physiological measurements may be performed to determine appropriate therapy by using imaging catheters or fractional flow reserve (FFR) measurements.

In PVI, the physician uses a robotic system to deliver the therapy and restore blood flow with techniques similar to NVI. The distal tip of the guidewire is navigated past the lesion and a microcatheter may be used to provide adequate support for the guidewire for complex anatomies. The blood flow is restored by delivering and deploying a stent or balloon to the lesion. As with PCI, lesion preparation and diagnostic imaging may be used as well.

When support at the distal end of a catheter or guidewire is needed, for example, to navigate tortuous or calcified vasculature, to reach distal anatomical locations, or to cross hard lesions, an over-the-wire (OTW) catheter or coaxial system is used. An OTW catheter has a lumen for the guidewire that extends the full length of the catheter. This provides a relatively stable system because the guidewire is supported along the whole length. This system, however, has some disadvantages, including higher friction, and longer overall length compared to rapid-exchange catheters (see below). Typically to remove or exchange an OTW catheter while maintaining the position of the indwelling guidewire, the exposed length (outside of the patient) of guidewire must be longer than the OTW catheter. A 300 cm long guidewire is typically sufficient for this purpose and is often referred to as an exchange length guidewire. Due to the length of the guidewire, two operators are needed to remove or exchange an OTW catheter. This becomes even more challenging if a triple coaxial, known in the art as a tri-axial system, is used (quadruple coaxial catheters have also been known to be used). However, due to its stability, an OTW system is often used in NVI and PVI procedures. On the other hand, PCI procedures often use rapid exchange (or monorail) catheters. The guidewire lumen in a rapid exchange catheter runs only through a distal section of the catheter, called the monorail or rapid exchange (RX) section. With a RX system, the operator manipulates the interventional devices parallel to each other (as opposed to with an OTW system, in which the devices are manipulated in a serial configuration), and the exposed length of guidewire only needs to be slightly longer than the RX section of the catheter. A rapid exchange length guidewire is typically 180-200 cm long. Given the shorter length guidewire and monorail, RX catheters can be exchanged by a single operator. However, RX catheters are often inadequate when more distal support is needed.

### SUMMARY

In accordance with an embodiment, a robotic drive system for a catheter-based procedure system includes a positioning system coupled to a patient table, the patient table having a front side and a rear side. The rear side of the patient table has a rail. The robotic drive system further includes a linear member coupled to the positioning system at a connection point and at least three device modules coupled to the linear member. Each device module is independently controllable and includes a drive module having a front side and a cassette mounted on the drive module. The cassette has a front side and is configured to support an elongated medical device having a longitudinal device axis. The cassette is mounted on the drive module in a vertical orientation so that the front side of the cassette is parallel to the front side of the drive. In addition, a width defined between the longitudinal device axis of the elongated medical device and the connection point of the linear member to the positioning system is equal to or less than a distance between an insertion point for the elongated medical device to a patient and the rail on the rear side of the patient table.

In accordance with another embodiment, a robotic drive system for a catheter-based procedure system includes a linear member and at least one device module coupled to the linear member. The at least one device module is independently controllable and includes a drive module and a cassette mounted on the drive module. The drive module includes a housing having a front side including a recess, a motor having a shaft, the motor disposed within the housing and the shaft positioned in the recess of the front side of the housing, and capstan directly mounted to the motor shaft. The cassette has a front side and is configured to support an elongated medical device having a longitudinal device axis The cassette is mounted on the drive module in a vertical orientation so that the front side of the cassette is parallel to the front side of the drive module and the cassette is coupled to the capstan.

In accordance with another embodiment, a robotic drive system for a catheter-based procedure system includes a positioning system coupled to a patient table. The patient table has a front side and a rear side and the rear side of the patient table has a rail. The robotic drive system further includes a linear member coupled to the positioning system at a connection point. The linear member has a distal end and a proximal end. The robotic system further includes at least three device modules coupled to the linear member. Each device module is independently controllable and is configured to support an elongated medical device having a longitudinal device axis. The positioning system is configured to position the linear member and the at least three device modules at a pitch angle defined between a horizontal axis parallel to the patient table and the proximal end of the linear member. The pitch angle is less than 10 degrees.

### BRIEF DESCRIPTION OF THE DRAWINGS

The invention will become more fully understood from the following detailed description, taken in conjunction with the accompanying drawings, wherein the reference numerals refer to like parts in which:

FIG. 1 is a perspective view of an exemplary catheter-based procedure system in accordance with an embodiment;

FIG. 2 is a schematic block diagram of an exemplary catheter-based procedure system in accordance with an embodiment;

FIG. 3 is a perspective view of a robotic drive for a catheter-based procedure system in accordance with an embodiment;

FIG. 4 is a diagram illustrating an elongated medical device axis of manipulation and the introductory point into the patient;

FIGS. 5a and 5b are diagrams illustrating the effect of the thickness of a robotic drive on the loss of working length;

FIG. 6 is a diagram illustrating an exemplary orientation to minimize loss of working length;

FIG. 7 is a perspective view of a device module with a vertically mounted cassette in accordance with an embodiment;

FIG. 8 is a rear perspective view of a device module with a vertically mounted cassette in accordance with an embodiment;

FIG. 9 is a front view of a distal end of a device module with a vertically mounted cassette in accordance with an embodiment;

FIG. 10 is a front view of a distal end of a device module with a horizontally mounted cassette in accordance with an embodiment;

FIG. 11a is a perspective view of a robotic drive with vertically mounted device modules in accordance with an embodiment;

FIG. 11b is a perspective view of a rack and pinion drive mechanism for a single drive module of a robotic drive in accordance with an embodiment;

FIG. 12 is a front view of a robotic drive with vertically mounted device modules in accordance with an embodiment;

FIG. 13 is a front view of a robotic drive with vertically mounted device modules in accordance with an embodiment;

FIG. 14 is a front view of an example cassette and elongated medical device in accordance with an embodiment;

FIG. 15 is a perspective view of a bedside unit of a catheter-based procedure system mounted on a patient table in accordance with an embodiment;

FIG. 16 is a top view of a bedside unit of a catheter-based procedure system mounted on a patient table in accordance with an embodiment;

FIG. 17 is a front view a bedside unit of a catheter-based procedure system mounted on a patient table in accordance with an embodiment;

FIG. 18 is a rear cross sectional view of a robotic drive with vertically mounted device modules in accordance with an embodiment;

FIG. 19a is a perspective view of a drive module in accordance with an embodiment;

FIG. 19b is a front view of a motor shaft in a recess of a drive module in accordance with an embodiment;

FIG. 19c is a perspective view of a capstan for a coupler of a drive module in accordance with an embodiment;

FIG. 20 is a front view of a robotic drive illustrating a pitch angle in accordance with an embodiment; and

FIG. 21 is a top view of a bedside unit of a catheter-based procedure system mounted on a patient table in accordance with an embodiment.

### DETAILED DESCRIPTION

The following definitions will be used herein. The term elongated medical device (EMD) refers to, but is not limited to, catheters (e.g. guide catheters, microcatheters, balloon/stent catheters), wire-based devices (guidewires, embolization coils, stent retrievers, etc.), and devices that have a combination of these. Wire-based EMD includes, but is not limited to, guidewires, microwires, a proximal pusher for embolization coils, stent retrievers, self-expanding stents, and flow divertors. Typically wire-based EMD's do not have a hub or handle at its proximal terminal end. In one embodiment the EMD is a catheter having a hub at a proximal end of the catheter and a flexible shaft extending from the hub toward the distal end of the catheter, wherein the shaft is more flexible than the hub. In one embodiment the catheter includes an intermediary portion that transitions between the hub and the shaft that has an intermediate flexibility that is less rigid than the hub and more rigid than the shaft. In one embodiment the intermediary portion is a strain relief.

The terms distal and proximal define relative locations of two different features. With respect to a robotic drive the terms distal and proximal are defined by the position of the robotic drive in its intended use relative to a patient. When used to define a relative position, the distal feature is the feature of the robotic drive that is closer to the patient than a proximal feature when the robotic drive is in its intended in-use position. Within a patient, any vasculature landmark further away along the path from the access point is considered more distal than a landmark closer to the access point, where the access point is the point at which the EMD enters the patient. Similarly, the proximal feature is the feature that is farther from the patient than the distal feature when the robotic drive in its intended in-use position. When used to define direction, the distal direction refers to a path on which something is moving or is aimed to move or along which something is pointing or facing from a proximal feature toward a distal feature and/or patient when the robotic drive is in its intended in-use position. The proximal direction is the opposite direction of the distal direction.

The term longitudinal axis of a member (e.g., an EMD or other element in the catheter-based procedure system) is the direction of orientation going from a proximal portion of the member to a distal portion of the member. By way of example, the longitudinal axis of a guidewire is the direction of orientation from a proximal portion of the guide wire toward a distal portion of the guidewire even though the guidewire may be non-linear in the relevant portion. The term axial movement of a member refers to translation of the member along the longitudinal axis of the member. When a distal end of an EMD is axially moved in a distal direction along its longitudinal axis into or further into the patient, the EMD is being advanced. When the distal end of an EMD is axially moved in a proximal direction along its longitudinal axis out of or further out of the patient, the EMD is being withdrawn. The term rotational movement of a member refers to change in angular orientation of the member about the local longitudinal axis of the member. Rotational movement of an EMD corresponds to clockwise or counterclockwise rotation of the EMD about its longitudinal axis due to an applied torque.

The term axial insertion refers to inserting a first member into a second member along the longitudinal axes of the second member. The term lateral insertion refers to inserting a first member into a second member along a direction in a plane perpendicular to the longitudinal axis of the second member. This can also be referred to as radial loading or side loading. The term pinch refers to releasably fixing an EMD to a member such that the EMD and member move together when the member moves. The term unpinch refers to releasing the EMD from a member such that the EMD and member move independently when the member moves. The term clamp refers to releasably fixing an EMD to a member such that the EMD's movement is constrained with respect to the member. The member can be fixed with respect to a global coordinate system or with respect to a local coordinate system. The term unclamp refers to releasing the EMD from the member such that the EMD can move independently.

The term grip refers to the application of a force or torque to an EMD by a drive mechanism that causes motion of the EMD without slip in at least one degree of freedom. The term ungrip refers to the release of the application of force or torque to the EMD by a drive mechanism such that the position of the EMD is no longer constrained. In one example, an EMD gripped between two tires will rotate about its longitudinal axis when the tires move longitudinally relative to one another. The rotational movement of the EMD is different than the movement of the two tires. The position of an EMD that is gripped is constrained by the drive mechanism. The term buckling refers to the tendency of a flexible EMD when under axial compression to bend away from the longitudinal axis or intended path along which it is being advanced. In one embodiment axial compression occurs in response to resistance from being navigated in the vasculature. The distance an EMD may be driven along its longitudinal axis without support before the EMD buckles is referred to herein as the device buckling distance. The device buckling distance is a function of the device's stiffness, geometry (including but not limited to diameter), and force being applied to the EMD. Buckling may cause the EMD to form an arcuate portion different than the intended path. Kinking is a case of buckling in which deformation of the EMD is non-elastic resulting in a permanent set.

The terms top, up, upper, and above refer to the general direction away from the direction of gravity and the terms bottom, down, lower, and below refer to the general direction in the direction of gravity. The term inwardly refers to the inner portion of a feature. The term outwardly refers to the outer portion of a feature. The term front refers to the side of the robotic drive (or an element of the robotic drive or other element of the catheter procedure system) that faces a bedside user and away from the positioning system, such as an articulating arm. The term rear refers to the side of the robotic drive (or an element of the robotic drive or other element of the catheter procedure system) that is closest to the positioning system, such as the articulating arm. The term sterile interface refers to an interface or boundary between a sterile and non-sterile unit. For example, a cassette may be a sterile interface between the robotic drive and at least one EMD. The term sterilizable unit refers to an apparatus that is capable of being sterilized (free from pathogenic microorganisms). This includes, but is not limited to, a cassette, consumable unit, drape, device adapter, and sterilizable drive modules/units (which may include electromechanical components). Sterilizable Units may come into contact with the patient, other sterile devices, or anything else placed within the sterile field of a medical procedure.

The term on-device adapter refers to sterile apparatus capable of releasably pinching an EMD to provide a driving interface. For example, the on-device adapter is also known as an end-effector or EMD capturing device. In one non-limiting embodiment, the on-device adapter is a collet that is operatively controlled robotically to rotate the EMD about its longitudinal axis, to pinch and/or unpinch the EMD to the collet, and/or to translate the EMD along its longitudinal axis. In one embodiment the on-device adapter is a hub-drive mechanism such as a driven gear located on the hub of an EMD.

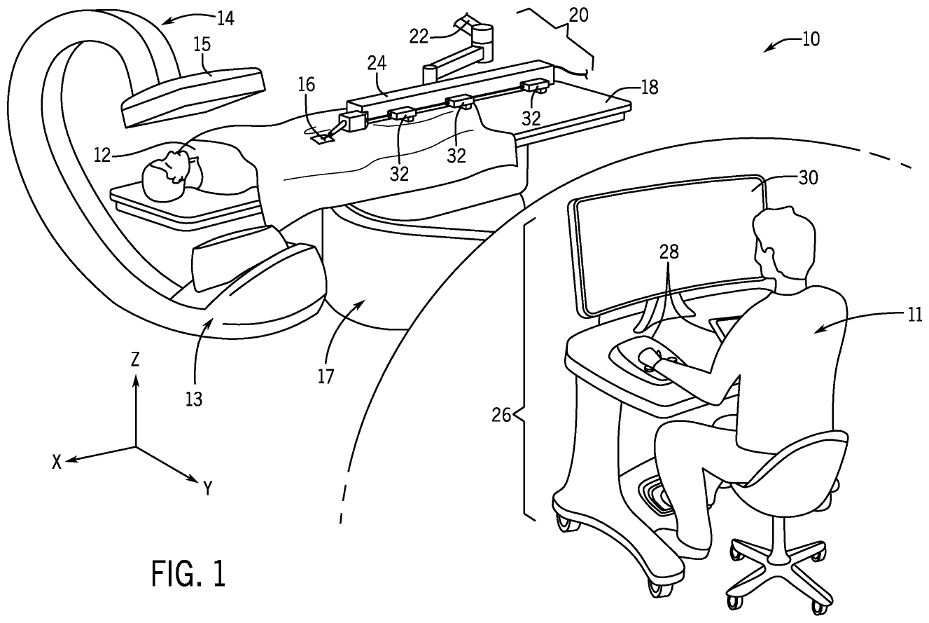

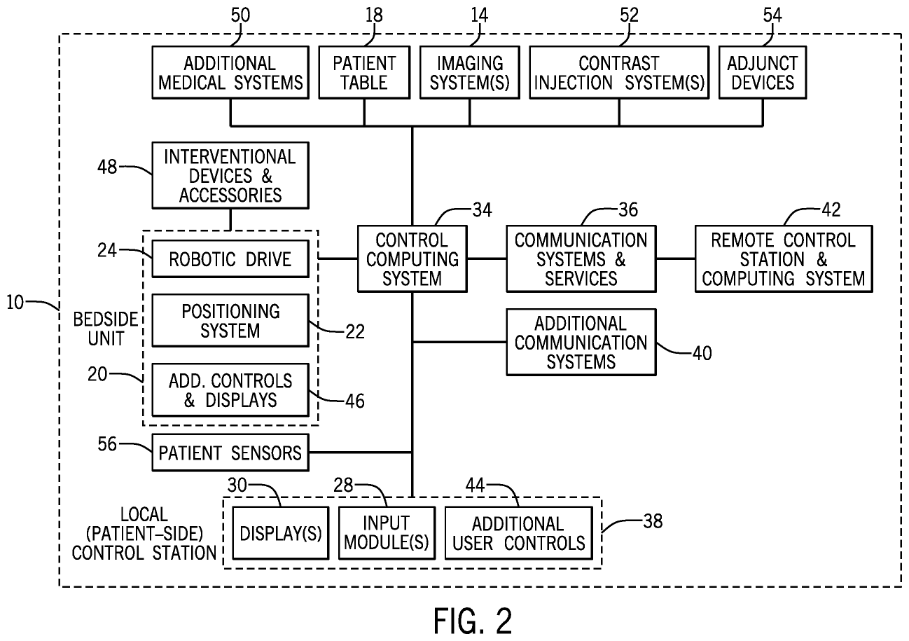

FIG. 1 is a perspective view of an exemplary catheter-based procedure system 10 in accordance with an embodiment. Catheter-based procedure system 10 may be used to perform catheter-based medical procedures, e.g., percutaneous intervention procedures such as a percutaneous coronary intervention (PCI) (e.g., to treat STEMI), a neurovascular interventional procedure (NVI) (e.g., to treat an emergent large vessel occlusion (ELVO)), peripheral vascular intervention procedures (PVI) (e.g., for critical limb ischemia (CLI), etc.). Catheter-based medical procedures may include diagnostic catheterization procedures during which one or more catheters or other elongated medical devices (EMDs) are used to aid in the diagnosis of a patient's disease. For example, during one embodiment of a catheter-based diagnostic procedure, a contrast media is injected onto one or more arteries through a catheter and an image of the patient's vasculature is taken. Catheter-based medical procedures may also include catheter-based therapeutic procedures (e.g., angioplasty, stent placement, treatment of peripheral vascular disease, clot removal, arterial venous malformation therapy, treatment of aneurysm, etc.) during which a catheter (or other EMD) is used to treat a disease. Therapeutic procedures may be enhanced by the inclusion of adjunct devices 54 (shown in FIG. 2) such as, for example, intravascular ultrasound (IVUS), optical coherence tomography (OCT), fractional flow reserve (FFR), etc. It should be noted, however, that one skilled in the art would recognize that certain specific percutaneous intervention devices or components (e.g., type of guidewire, type of catheter, etc.) may be selected based on the type of procedure that is to be performed. Catheter-based procedure system 10 can perform any number of catheter-based medical procedures with minor adjustments to accommodate the specific percutaneous intervention devices to be used in the procedure.

Catheter-based procedure system 10 includes, among other elements, a bedside unit 20 and a control station 26. Bedside unit 20 includes a robotic drive 24 and a positioning system 22 that are located adjacent to a patient 12. Patient 12 is supported on a patient table 18. The positioning system 22 is used to position and support the robotic drive 24. The positioning system 22 may be, for example, a robotic arm, an articulated arm, a holder, etc. The positioning system 22 may be attached at one end to, for example, a rail on the patient table 18, a base, or a cart. The other end of the positioning system 22 is attached to the robotic drive 24. The positioning system 22 may be moved out of the way (along with the robotic drive 24) to allow for the patient 12 to be placed on the patient table 18. Once the patient 12 is positioned on the patient table 18, the positioning system 22 may be used to situate or position the robotic drive 24 relative to the patient 12 for the procedure. In an embodiment, patient table 18 is operably supported by a pedestal 17, which is secured to the floor and/or earth. Patient table 18 is able to move with multiple degrees of freedom, for example, roll, pitch, and yaw, relative to the pedestal 17. Bedside unit 20 may also include controls and displays 46 (shown in FIG. 2). For example, controls and displays may be located on a housing of the robotic drive 24.

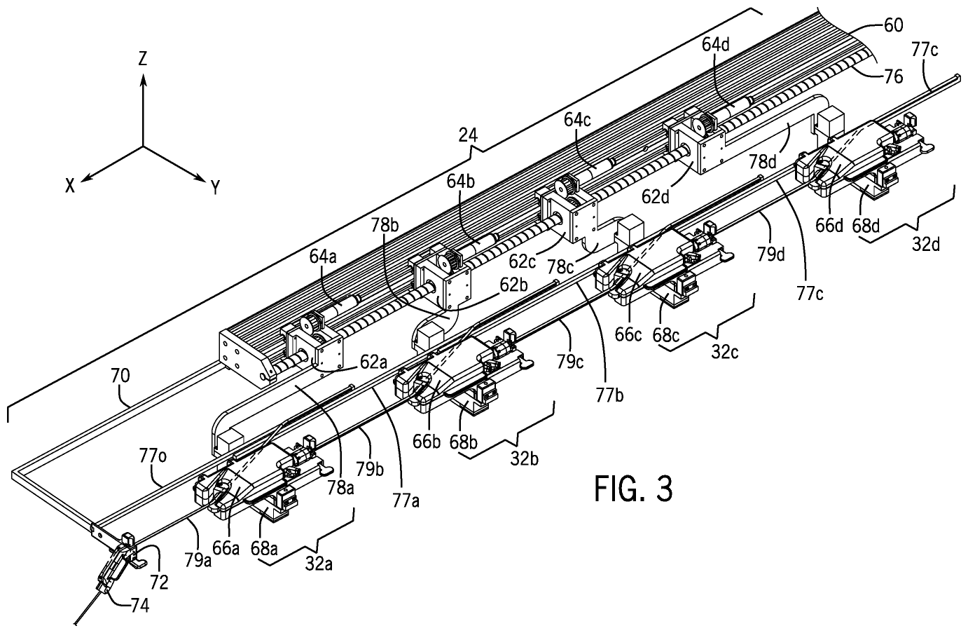

Generally, the robotic drive 24 may be equipped with the appropriate percutaneous interventional devices and accessories 48 (shown in FIG. 2) (e.g., guidewires, various types of catheters including balloon catheters, stent delivery systems, stent retrievers, embolization coils, liquid embolics, aspiration pumps, device to deliver contrast media, medicine, hemostasis valve adapters, syringes, stopcocks, inflation device, etc.) to allow the user or operator 11 to perform a catheter-based medical procedure via a robotic system by operating various controls such as the controls and inputs located at the control station 26. Bedside unit 20, and in particular robotic drive 24, may include any number and/or combination of components to provide bedside unit 20 with the functionality described herein. A user or operator 11 at control station 26 is referred to as the control station user or control station operator and referred to herein as user or operator. A user or operator at bedside unit 20 is referred to as bedside unit user or bedside unit operator. The robotic drive 24 includes a plurality of device modules 32a-d mounted to a rail or linear member 60 (shown in FIG. 3). The rail or linear member 60 guides and supports the device modules. Each of the device modules 32a-d may be used to drive an EMD such as a catheter or guidewire. For example, the robotic drive 24 may be used to automatically feed a guidewire into a diagnostic catheter and into a guide catheter in an artery of the patient 12. One or more devices, such as an EMD, enter the body (e.g., a vessel) of the patient 12 at an insertion point 16 via, for example, an introducer sheath.

Bedside unit 20 is in communication with control station 26, allowing signals generated by the user inputs of control station 26 to be transmitted wirelessly or via hardwire to bedside unit 20 to control various functions of bedside unit 20. As discussed below, control station 26 may include a control computing system 34 (shown in FIG. 2) or be coupled to the bedside unit 20 through a control computing system 34. Bedside unit 20 may also provide feedback signals (e.g., loads, speeds, operating conditions, warning signals, error codes, etc.) to control station 26, control computing system 34 (shown in FIG. 2), or both. Communication between the control computing system 34 and various components of the catheter-based procedure system 10 may be provided via a communication link that may be a wireless connection, cable connections, or any other means capable of allowing communication to occur between components. Control station 26 or other similar control system may be located either at a local site (e.g., local control station 38 shown in FIG. 2) or at a remote site (e.g., remote control station and computer system 42 shown in FIG. 2). Catheter procedure system 10 may be operated by a control station at the local site, a control station at a remote site, or both the local control station and the remote control station at the same time. At a local site, user or operator 11 and control station 26 are located in the same room or an adjacent room to the patient 12 and bedside unit 20. As used herein, a local site is the location of the bedside unit 20 and a patient 12 or subject (e.g., animal or cadaver) and the remote site is the location of a user or operator 11 and a control station 26 used to control the bedside unit 20 remotely. A control station 26 (and a control computing system) at a remote site and the bedside unit 20 and/or a control computing system at a local site may be in communication using communication systems and services 36 (shown in FIG. 2), for example, through the Internet. In an embodiment, the remote site and the local (patient) site are away from one another, for example, in different rooms in the same building, different buildings in the same city, different cities, or other different locations where the remote site does not have physical access to the bedside unit 20 and/or patient 12 at the local site.

Control station 26 generally includes one or more input modules 28 configured to receive user inputs to operate various components or systems of catheter-based procedure system 10. In the embodiment shown, control station 26 allows the user or operator 11 to control bedside unit 20 to perform a catheter-based medical procedure. For example, input modules 28 may be configured to cause bedside unit 20 to perform various tasks using percutaneous intervention devices (e.g., EMDs) interfaced with the robotic drive 24 (e.g., to advance, retract, or rotate a guidewire, advance, retract or rotate a catheter, inflate or deflate a balloon located on a catheter, position and/or deploy a stent, position and/or deploy a stent retriever, position and/or deploy a coil, inject contrast media into a catheter, inject liquid embolics into a catheter, inject medicine or saline into a catheter, aspirate on a catheter, or to perform any other function that may be performed as part of a catheter-based medical procedure). Robotic drive 24 includes various drive mechanisms to cause movement (e.g., axial and rotational movement) of the components of the bedside unit 20 including the percutaneous intervention devices.

In one embodiment, input modules 28 may include one or more touch screens, joysticks, scroll wheels, and/or buttons. In addition to input modules 28, the control station 26 may use additional user controls 44 (shown in FIG. 2) such as foot switches and microphones for voice commands, etc. Input modules 28 may be configured to advance, retract, or rotate various components and percutaneous intervention devices such as, for example, a guidewire, and one or more catheters or microcatheters. Buttons may include, for example, an emergency stop button, a multiplier button, device selection buttons and automated move buttons. When an emergency stop button is pushed, the power (e.g., electrical power) is shut off or removed to bedside unit 20. When in a speed control mode, a multiplier button acts to increase or decrease the speed at which the associated component is moved in response to a manipulation of input modules 28. When in a position control mode, a multiplier button changes the mapping between input distance and the output commanded distance. Device selection buttons allow the user or operator 11 to select which of the percutaneous intervention devices loaded into the robotic drive 24 are controlled by input modules 28. Automated move buttons are used to enable algorithmic movements that the catheter-based procedure system 10 may perform on a percutaneous intervention device without direct command from the user or operator 11. In one embodiment, input modules 28 may include one or more controls or icons (not shown) displayed on a touch screen (that may or may not be part of a display 30), that, when activated, causes operation of a component of the catheter-based procedure system 10. Input modules 28 may also include a balloon or stent control that is configured to inflate or deflate a balloon and/or deploy a stent. Each of the input modules 28 may include one or more buttons, scroll wheels, joysticks, touch screen, etc. that may be used to control the particular component or components to which the control is dedicated. In addition, one or more touch screens may display one or more icons (not shown) related to various portions of input modules 28 or to various components of catheter-based procedure system 10.

Control station 26 may include a display 30. In other embodiments, the control station 26 may include two or more displays 30. Display 30 may be configured to display information or patient specific data to the user or operator 11 located at control station 26. For example, display 30 may be configured to display image data (e.g., X-ray images, MRI images, CT images, ultrasound images, etc.), hemodynamic data (e.g., blood pressure, heart rate, etc.), patient record information (e.g., medical history, age, weight, etc.), lesion or treatment assessment data (e.g., IVUS, OCT, FFR, etc.). In addition, display 30 may be configured to display procedure specific information (e.g., procedural checklist, recommendations, duration of procedure, catheter or guidewire position, volume of medicine or contrast agent delivered, etc.). Further, display 30 may be configured to display information to provide the functionalities associated with control computing system 34 (shown in FIG. 2). Display 30 may include touch screen capabilities to provide some of the user input capabilities of the system.

Catheter-based procedure system 10 also includes an imaging system 14. Imaging system 14 may be any medical imaging system that may be used in conjunction with a catheter based medical procedure (e.g., non-digital X-ray, digital X-ray, CT, MRI, ultrasound, etc.). In an exemplary embodiment, imaging system 14 is a digital X-ray imaging device that is in communication with control station 26. In one embodiment, imaging system 14 may include a C-arm (shown in FIG. 1) that allows imaging system 14 to partially or completely rotate around patient 12 in order to obtain images at different angular positions relative to patient 12 (e.g., sagittal views, caudal views, anterior-posterior views, etc.). In one embodiment imaging system 14 is a fluoroscopy system including a C-arm having an X-ray source 13 and a detector 15, also known as an image intensifier.

Imaging system 14 may be configured to take X-ray images of the appropriate area of patient 12 during a procedure. For example, imaging system 14 may be configured to take one or more X-ray images of the head to diagnose a neurovascular condition. Imaging system 14 may also be configured to take one or more X-ray images (e.g., real time images) during a catheter-based medical procedure to assist the user or operator 11 of control station 26 to properly position a guidewire, guide catheter, microcatheter, stent retriever, coil, stent, balloon, etc. during the procedure. The image or images may be displayed on display 30. For example, images may be displayed on display 30 to allow the user or operator 11 to accurately move a guide catheter or guidewire into the proper position.

In order to clarify directions, a rectangular coordinate system is introduced with X, Y, and Z axes. The positive X axis is oriented in a longitudinal (axial) distal direction, that is, in the direction from the proximal end to the distal end, stated another way from the proximal to distal direction. The Y and Z axes are in a transverse plane to the X axis, with the positive Z axis oriented up, that is, in the direction opposite of gravity, and the Y axis is automatically determined by right-hand rule.

FIG. 2 is a block diagram of catheter-based procedure system 10 in accordance with an exemplary embodiment. Catheter-procedure system 10 may include a control computing system 34. Control computing system 34 may physically be, for example, part of control station 26 (shown in FIG. 1). Control computing system 34 may generally be an electronic control unit suitable to provide catheter-based procedure system 10 with the various functionalities described herein. For example, control computing system 34 may be an embedded system, a dedicated circuit, a general-purpose system programmed with the functionality described herein, etc. Control computing system 34 is in communication with bedside unit 20, communications systems and services 36 (e.g., Internet, firewalls, cloud services, session managers, a hospital network, etc.), a local control station 38, additional communications systems 40 (e.g., a telepresence system), a remote control station and computing system 42, and patient sensors 56 (e.g., electrocardiogram (ECG) devices, electroencephalogram (EEG) devices, blood pressure monitors, temperature monitors, heart rate monitors, respiratory monitors, etc.). The control computing system is also in communication with imaging system 14, patient table 18, additional medical systems 50, contrast injection systems 52 and adjunct devices 54 (e.g., IVUS, OCT, FFR, etc.). The bedside unit 20 includes a robotic drive 24, a positioning system 22 and may include additional controls and displays 46. As mentioned above, the additional controls and displays may be located on a housing of the robotic drive 24. Interventional devices and accessories 48 (e.g., guidewires, catheters, etc.) interface to the bedside system 20. In an embodiment, interventional devices and accessories 48 may include specialized devices (e.g., IVUS catheter, OCT catheter, FFR wire, diagnostic catheter for contrast, etc.) which interface to their respective adjunct devices 54, namely, an IVUS system, an OCT system, and FFR system, etc.

In various embodiments, control computing system 34 is configured to generate control signals based on the user's interaction with input modules 28 (e.g., of a control station 26 (shown in FIG. 1) such as a local control station 38 or a remote control station 42) and/or based on information accessible to control computing system 34 such that a medical procedure may be performed using catheter-based procedure system 10. The local control station 38 includes one or more displays 30, one or more input modules 28, and additional user controls 44. The remote control station and computing system 42 may include similar components to the local control station 38. The remote 42 and local 38 control stations can be different and tailored based on their required functionalities. The additional user controls 44 may include, for example, one or more foot input controls. The foot input control may be configured to allow the user to select functions of the imaging system 14 such as turning on and off the X-ray and scrolling through different stored images. In another embodiment, a foot input device may be configured to allow the user to select which devices are mapped to scroll wheels included in input modules 28. Additional communication systems 40 (e.g., audio conference, video conference, telepresence, etc.) may be employed to help the operator interact with the patient, medical staff (e.g., angio-suite staff), and/or equipment in the vicinity of the bedside.

Catheter-based procedure system 10 may be connected or configured to include any other systems and/or devices not explicitly shown. For example, catheter-based procedure system 10 may include image processing engines, data storage and archive systems, automatic balloon and/or stent inflation systems, medicine injection systems, medicine tracking and/or logging systems, user logs, encryption systems, systems to restrict access or use of catheter-based procedure system 10, etc.

As mentioned, control computing system 34 is in communication with bedside unit 20 which includes a robotic drive 24, a positioning system 22 and may include additional controls and displays 46, and may provide control signals to the bedside unit 20 to control the operation of the motors and drive mechanisms used to drive the percutaneous intervention devices (e.g., guidewire, catheter, etc.). The various drive mechanisms may be provided as part of a robotic drive 24. FIG. 3 is a perspective view of a robotic drive for a catheter-based procedure system 10 in accordance with an embodiment. In FIG. 3, a robotic drive 24 includes multiple device modules 32a-d coupled to a linear member 60. Each device module 32a-d is coupled to the linear member 60 via a stage 62a-d moveably mounted to the linear member 60. A device module 32a-d may be connected to a stage 62a-d using a connector such as an offset bracket 78a-d. In another embodiment, the device module 32a-d is directly mounted to the stage 62a-d. Each stage 62a-d may be independently actuated to move linearly along the linear member 60. Accordingly, each stage 62a-d (and the corresponding device module 32a-d coupled to the stage 62a-d) may independently move relative to each other and the linear member 60. A drive mechanism is used to actuate each stage 62a-d. In the embodiment shown in FIG. 3, the drive mechanism includes independent stage translation motors 64a-d coupled to each stage 62a-d and a stage drive mechanism 76, for example, a lead screw via a rotating nut, a rack via a pinion, a belt via a pinion or pulley, a chain via a sprocket, or the stage translation motors 64a-d may be linear motors themselves. In some embodiments, the stage drive mechanism 76 may be a combination of these mechanisms, for example, each stage 62a-d could employ a different type of stage drive mechanism. In an embodiment where the stage drive mechanism is a screw (e.g., a lead screw, a ball screw or any type of screw mechanism) and rotating nut, the lead screw may be rotated and each stage 62a-d may engage and disengage from the lead screw to move, e.g., to advance or retract. In the embodiment shown in FIG. 3, the stages 62a-d and device modules 32a-d are in a serial drive configuration.

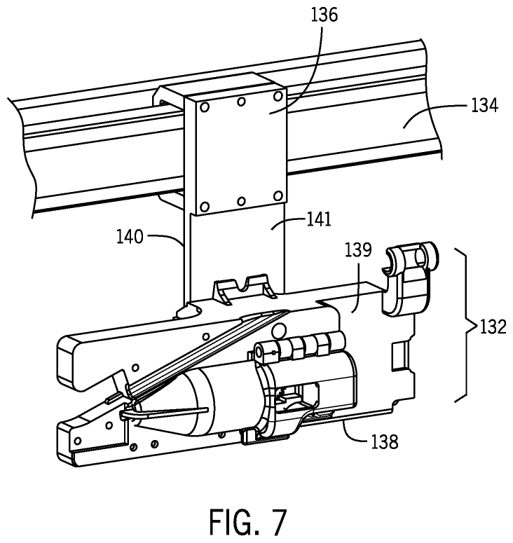

Each device module 32a-d includes a drive module 68a-d and a cassette 66a-d mounted on and coupled to the drive module 68a-d. In the embodiment shown in FIG. 3, each cassette 66a-d is mounted to the drive module 68a-d in an orientation such that the cassette 66a-d is mounted on a drive module 68a-d by moving the cassette 66a-d in a vertical direction down onto the drive module 66a-d. A top face or side of the cassette 66a-d is parallel to a top face or side (i.e., a mounting surface) of the drive module 68a-d when the cassette 66a-d is mounted on the drive module 68a-d. As used herein, the mounting orientation shown in FIG. 3 is referred to as a horizontal orientation. In other embodiments, each cassette 66a-d may be mounted to the drive module 68a-d in other mounting orientations. Various mounting orientations are described further below with respect to FIGS. 7-10. Each cassette 66a-d is configured to interface with and support a proximal portion of an EMD (not shown). In addition, each cassette 66a-d may include elements to provide one or more degrees of freedom in addition to the linear motion provided by the actuation of the corresponding stage 62a-d to move linearly along the linear member 60. For example, the cassette 66a-d may include elements that may be used to rotate the EMD when the cassette is coupled to the drive module 68a-d. Each drive module 68a-d includes at least one coupler to provide a drive interface to the mechanisms in each cassette 66a-d to provide the additional degree of freedom. Each cassette 66a-d also includes a channel in which a device support 79a-d is positioned, and each device support 79a-d is used to prevent an EMD from buckling. A support arm 77a, 77b, and 77c is attached to each device module 32a, 32b, and 32c, respectively, to provide a fixed point for support of a proximal end of the device supports 79b, 79c, and 79d, respectively. The robotic drive 24 may also include a device support connection 72 connected to a device support 79, a distal support arm 70 and a support arm 77₀. Support arm 77₀ is used to provide a fixed point for support of the proximal end of the distal most device support 79a housed in the distal most device module 32a. In addition, an introducer interface support (redirector) 74 may be connected to the device support connection 72 and an EMD (e.g., an introducer sheath). The configuration of robotic drive 24 has the benefit of reducing volume and weight of the drive robotic drive 24 by using actuators on a single linear member.

To prevent contaminating the patient with pathogens, healthcare staff use aseptic technique in a room housing the bedside unit 20 and the patient 12 or subject (shown in FIG. 1). A room housing the bedside unit 20 and patient 12 may be, for example, a cath lab or an angio suite. Aseptic technique consists of using sterile barriers, sterile equipment, proper patient preparation, environmental controls and contact guidelines. Accordingly, all EMDs and interventional accessories are sterilized and can only be in contact with either sterile barriers or sterile equipment. In an embodiment, a sterile drape (not shown) is placed over the non-sterile robotic drive 24. Each cassette 66a-d is sterilized and acts as a sterile interface between the draped robotic drive 24 and at least one EMD. Each cassette 66a-d can be designed to be sterile for single use or to be re-sterilized in whole or part so that the cassette 66a-d or its components can be used in multiple procedures.

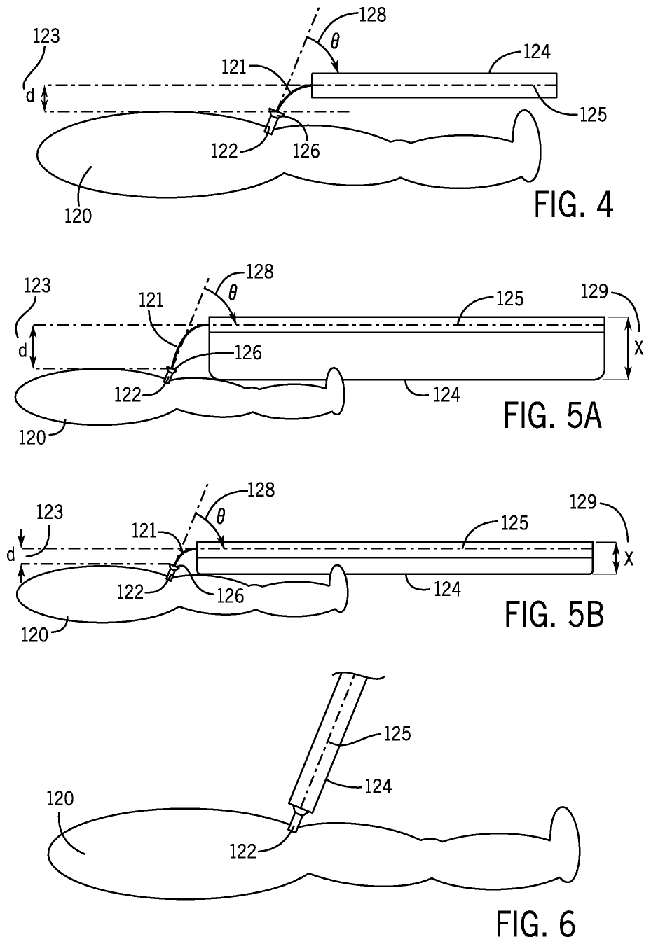

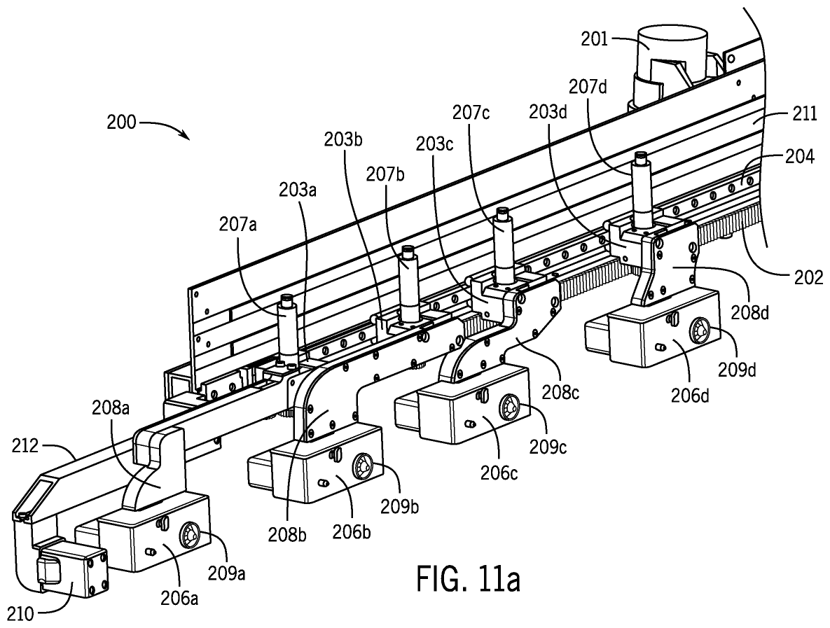

As shown in FIG. 1, one or more EMDs may enter the body of a patient (e.g., a vessel) at an insertion point 16 using, for example, an introducer and introducer sheath. The introducer sheath typically orients at an angle, usually less than 45 degrees, to the axis of the vessel in a patient 120 (shown in FIGS. 4-6). Any height difference between where the EMD enters the body (the introducer sheath's proximal opening 126 shown in FIG. 4) and the longitudinal drive axis of the robotic drive 124 will directly affect the working length for the elongated medical device. The more an elongated medical device needs to compensate for differences in displacement and angle, the less the elongated medical device will be able to enter the body when the robotic drive is at its maximum distal (forward) position. It is beneficial to have a robotic drive that is at the same height and angle as the introducer sheath. FIG. 4 is a diagram illustrating an elongated medical device axis of manipulation and the introductory point into the patient. FIG. 4 shows a height difference (d) 123 between the proximal end 126 of the introducer sheath 122 and the longitudinal device axis and an angular difference (θ) 128 between the introducer sheath 122 and the longitudinal device axis 125 of the robotic drive 124. The elongated medical device 121 is constrained on each axis and creates a curve with tangentially aligned end points. The length of this curve represents a length of the elongated medical device 121 that cannot be driven any further forward by the robotic drive 124 and cannot enter the introducer sheath 122 due to the misalignment. A higher angle (θ) 128 also leads to higher device friction. In general, lower angular misalignment (θ) 128, and linear misalignment d 123 can lead to reduced friction and reduced loss of working length. While FIG. 10 illustrates a simplified example illustrating one linear and one rotational offset, it should be understood that this problem occurs in three dimensions, namely, three linear offsets and three rotational offsets. The thickness of the robotic drive 124 also plays a role in determining the location of the longitudinal device axis 125 relative to the introducer sheath 122.

FIGS. 5a and 5b are diagrams illustrating the effect of the thickness of a drive module, or robotic drive as a whole, on the loss of working length. FIG. 5a shows the location of the longitudinal device axis 125 of a robotic drive 124 relative to the introducer sheath 122, indicated by d 123, when the robotic drive 124 is thick as shown by the distance (X) 129 between an upper surface and a bottom surface of the robotic drive 124. FIG. 5b shows the location of the longitudinal device axis 125 of a robotic drive 124 relative to the introducer sheath 122, indicated by a shorter d 123, 'when the robotic drive 124 is shallow as shown by the distance (X) 129 between an upper surface and a bottom surface of the robotic drive 124. Reducing the thickness of the robotic drive 124 to get close to the patient and introducer sheath reduces the distance 123 between introducer sheath axis and device axis and reduces the loss of working length of the elongated medical device. FIG. 6 is a diagram illustrating an exemplary orientation to minimize loss of working length. In FIG. 6, the robotic drive is positioned to align the longitudinal device axis 125 of the robotic drive 124 to that of the introducer sheath 122. This eliminates loss of working length due to angular and linear misalignment of the elongated medical device. However, this position for the robotic drive 124 may not be practical due to the length and size of the robotic drive 124. Orienting a robotic drive at a sharp angle also affects the usability by making it difficult to load and unload elongated medical devices, and adjust and handle the robotic drive.

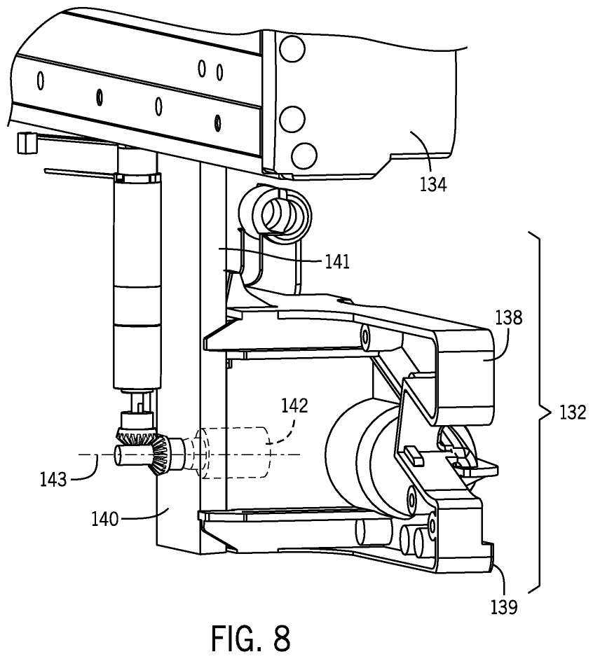

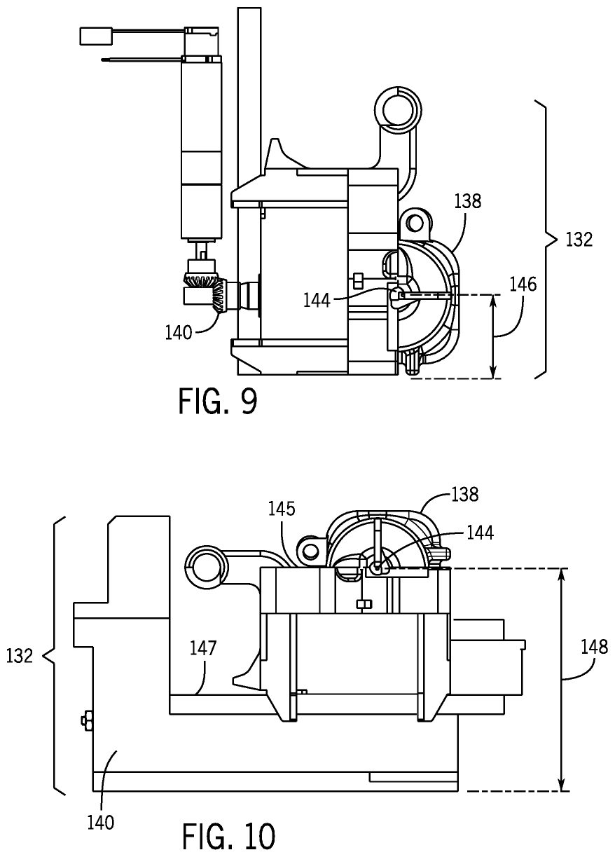

To reduce the distance between the robotic drive and the patient and the distance between the longitudinal device axis of the robotic drive and the introducer sheath, the cassette 66a-d of a device module 32 (shown in FIG. 3) may be mounted to the drive module 68a-d in an orientation such that the cassette 66a-d is mounted on a drive module 68a-d by moving the cassette 66a-d in a horizontal direction onto the drive module 66a-d. FIG. 7 is a perspective view of a device module with a vertically mounted cassette in accordance with an embodiment and FIG. 8 is a rear perspective view of a device module with a vertically mounted cassette in accordance with an embodiment. In FIGS. 7 and 8, a device module 132 includes a cassette 138 that is mounted to a drive nodule 140 such that a front face or side 139 of the cassette 138 is parallel to a front face or side 141 (i.e., a mounting surface) of the drive module 140. As used herein, the mounting orientation shown in FIGS. 7 and 8 is referred to as a vertical orientation. The device module 132 is connected to a stage 136 that is moveably mounted to a rail or linear member 134. The drive module 140 includes a coupler 142 that is used to provide a power interface to the cassette 138 to, for example, rotate an elongated medical device (not shown) positioned in the cassette. The coupler 142 rotates about an axis 143. As mentioned, the cassette 138 is mounted to the drive module 140 by moving the cassette 138 in a horizontal direction onto the mounting surface 141 so that the cassette is coupled to coupler 142 of the drive module 140. By mounting the cassette 138 vertically, the drive module 140 that the cassette 138 attaches to is located off to the side and no longer positioned between the cassette 138 and the patient. FIG. 9 is a front view of a distal end of a device module with a vertically mounted cassette in accordance with an embodiment. In FIG. 9, a distance 146 between the device axis of the elongated medical device 144 and the bottom surface of the device module 132 is shown. The vertical mounting orientation of the cassette 138 eliminates the need for the drive module 140 to be placed under the device axis and between the elongated medical device 144 and the patient. Rather, only a portion of the cassette 138 is positioned between the elongated medical device 138 and the patient. Vertically mounting the cassette 138 also reduces the distance 146 between the elongated medical device and the bottom surface of the device module 132 which allows the robotic drive to get closer to the patient and reduces loss of working length in an elongated medical device. By comparison, FIG. 10 is a front view of a distal end of a device module with a horizontally mounted cassette in accordance with an embodiment. In FIG. 10, a device module 132 is shown where the cassette 138 is horizontally mounted to a drive module 140. A top face or side 145 of the cassette 138 is parallel to a top face or side 147 (i.e., a mounting surface) of the drive module 140 when the cassette 138 is mounted on the drive module 140. The drive module 140 is under or below the cassette 138 and increases the distance 148 between the device axis of the elongated medical device 144 and the bottom surface of the device module 132. This can prevent the device axis from being as close to the introducer (not shown) as possible. A drive module 140 positioned under the cassette 138 may also interfere with the patient. In various other embodiment, a cassette may be mounted to the drive module at any angle. In yet another embodiment, the cassette may be mounted horizontally on an underside of the drive module to eliminate the need for a drive module between the device axis and the patient.

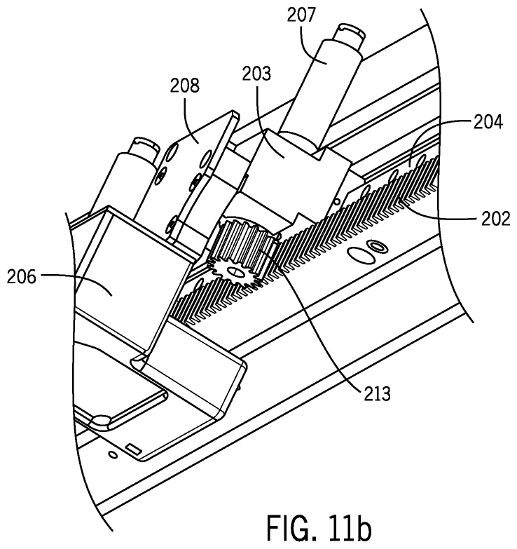

FIG. 11a is a perspective view of a robotic drive with vertically mounted device modules in accordance with an embodiment. In FIG. 11a, a robotic drive 200 includes multiple drive modules 206a-d coupled to a linear member 211. As discussed above, a cassette (not shown) may be mounted to each drive module 206a-d. In the robotic drive 200, each drive module 206a-d is configured so that a cassette may be mounted to a drive module 206a-d in a vertical orientation. As discussed above with respect to FIGS. 7-9, a vertical orientation of the drive modules 206a-d and the corresponding cassette (not shown) that is attached to each drive module 206a-d allows the drive 200 and the drive modules 206a-d to get closer to the patient and reduces the loss of working length in an EMD. Each drive module 206a-d includes at least one coupler 209a-d to provide a drive interface to mechanisms in each cassette to provide power to, for example, rotate an EMD using the mechanisms in the cassette. Each drive module 206a-d also includes a motor (not shown) that is used to rotate the coupler 209a-d. Each drive module 206a-d is coupled to the linear member 211 via a stage (or slide) 203a-d moveably mounted to the linear member 211 using, for example, a rail 204. A drive module 206a-d may be connected to a stage 203a-d using a connector such as an offset bracket 208a-d. In another embodiment, the drive module 206a-d may be directly mounted to the stage 203a-d. The robotic drive 211 may also include a device support connection 210 connected to a distal support arm 212. The distal support arm 212 extends away from the linear member 211 of the robotic drive 200 and may be attached to, for example, a frame of the robotic drive 200. The device support connection 210 and the distal support arm 212 are configured to provide a distal fixed point to support a distal end of a device support (not shown) in a cassette mounted to the most distal drive module 206a that is closest to the patient. The device support connection 210 may also be coupled to an introducer sheath hub (not shown).

Each stage 203a-d may be independently actuated to move linearly along the rail 204 of the linear member 211. Accordingly, each stage 203a-d (and the corresponding drive module 206a-d coupled to the stage 203a-d) may independently move relative to each other and the linear member 211. A drive mechanism is used to actuate each stage 203a-d. In the embodiment shown in FIG. 11a, the drive mechanism includes independent stage translation motors 207a-d coupled to each stage 203a-d and a stage drive mechanism. In FIG. 11a, the stage drive mechanism is a rack and pinion linear actuator mechanism that includes a rack 202 and a separate pinion (shown in FIG. 11b) for each stage 203a-d. The rail 204 is positioned above the rack 202 so that the rack 202 takes up the moment. FIG. 11b is a perspective view of a rack and pinion drive mechanism for a single drive module of a robotic drive in accordance with an embodiment. In FIG. 11b, the rack and pinion mechanism for a single drive module 206 (e.g., one of drive modules 206a-d shown in FIG. 11a) is shown. The drive module 206 is coupled to a stage 203 with an offset bracket 208. The stage 203 is movably coupled to a rail 204. In an embodiment, the stage 203 is configured to be as frictionless as possible. A pinion 213 is directly mounted to a motor 207, e.g., a shaft (not shown) of the motor 207. Known methods may be used to directly mount the pinion 213 directly to the motor shaft, e.g., a screw. Directly mounting the pinion 213 to the motor 207 shaft may reduce the height of the robotic drive 200 (shown in FIG. 11) as discussed further below. The pinion 213 meshes with the rack 202 (e.g., teeth of the pinion 213 mesh with the teeth of the rack 202). To actuate the stage 203 to move forward (i.e., in a distal direction towards the patient) along the rail 204, the pinion is rotated (e.g., in a counter clockwise direction when viewed from the bottom view shown in FIG. 11b) and moves along the rack 202 which pushes the drive module 206 forward while the rack 202 remains stationary. In an embodiment, the teeth of the rack 202 and the pinion 213 may be straight, helical, or other standard geometry.

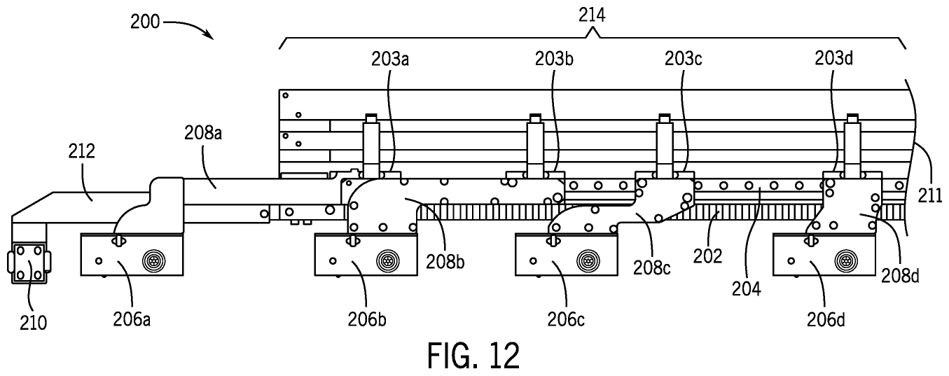

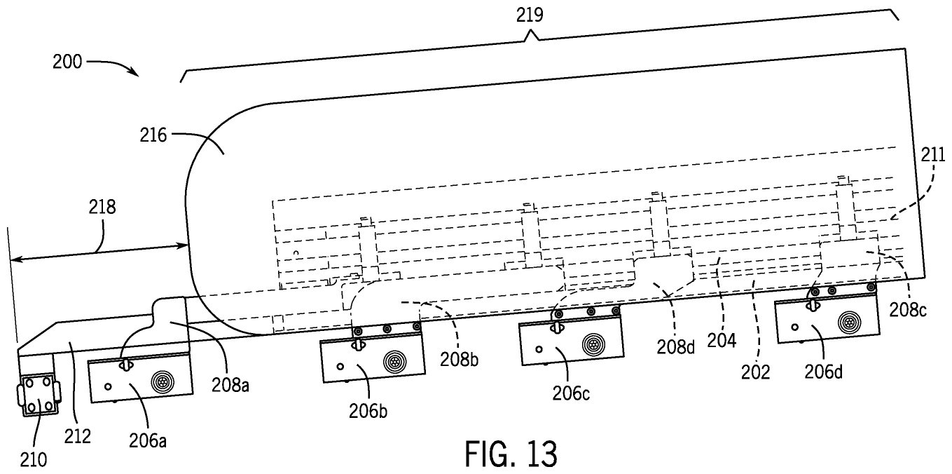

As mentioned, each drive module 206a-d may be connected to a stage 203a-d using a connector such as an offset bracket 208a-d. FIG. 12 is a front view of a robotic drive with vertically mounted device modules in accordance with an embodiment. Each drive module 206a-d is connected to an offset bracket 208a-d which is used to connect the drive module to a stage 203a-d. In order to reduce a length 214 of the linear member 211 of the robotic drive 200, the offset brackets 208a-d may be used to create offsets between a stage 203a-d and a drive module 206a-d (and a cassette (not shown) mounted to each drive module) to reduce gaps between stages 203a-d on the linear member 211 (e.g., on rail 204) when the drive modules are brought together. The length of each drive module 208a-d (and an associated cassette) may limit how close each stage may be brought to another stage on the rail 202 (e.g., how close stage 203b may be brought to stage 203a). The four stages 203a-d define an occupied rail length which affects the overall length 214 required for the rail 202 and the linear member 211. The occupied rail length and the overall length 214 of the rail 204 (and linear member 211) may be shortened by using offsets and offset brackets. Each offset bracket 208a-d defines an offset distance from the center of the respective stage 203a-d to which it is attached to a center of the drive module 206a-d attached to the stage 203a-d or a center of a cassette (not shown) attached to each drive module 206a-d. The offsets allow the stages 203a-d to be brought towards the center of the rail 204 which reduces the overall length of the robotic drive 200. In the embodiment shown in FIG. 11b, each offset bracket 208a-d positioned along rail 204 (and linear member 211) extends in a distal direction (i.e., forward facing) towards the patient. This configuration can allow the linear member 211 (and other elements of the robotic drive) to be farther away from an access site in the patient and an imaging system of the catheter procedure system. FIG. 13 is a front view of a robotic drive with vertically mounted device modules in accordance with an embodiment. In the embodiment of FIG. 13, the linear member 211 is disposed in a housing 216. The offsets created by the offset brackets 208a-d are used to minimize the length of the linear member and the required length 219 of the housing 216 by eliminating dead space between the stages (shown in FIG. 11a and 12). Accordingly, the length of the linear member 211 and the housing 216 for the linear member 211 may be minimized while the range of linear motion of the most distal drive module 206a can continue to move forward past the distal end of linear member 211 and housing 215 towards the patient. For example, the most distal drive module 206a can move past the distal end of the hosing 216 for a distance define by a length of the distal support arm 218. Advantageously, this allows the linear member 211 and housing 216 to avoid conflict with, for example, a C-arm (e.g., a detector 15 of a C-arm shown in FIG. 1) of an imaging system (e.g., a fluoroscopy imaging system). In addition, the use of offset brackets 208a-d can reduce the weight of the robotic drive 200 since the length of the linear member 211 (and a frame of the robotic drive) is minimized.

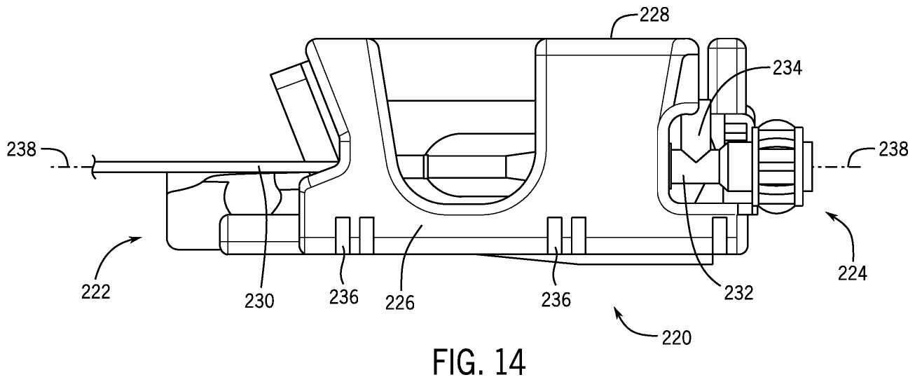

As mentioned above, a cassette may be mounted to each drive module 206a-d in the robotic drive 200. FIG. 14 is a front view of an example cassette and elongated medical device in accordance with an embodiment. Cassette 220 is configured for a vertical mount to a drive module (e.g., drive modules 206a-d shown in FIGS. 11a, 12 and 13) and includes features that enable the cassette 220 to be vertically mounted to a drive module (e.g., mounted in a vertical orientation as described above with respect to FIGS. 7-9) 9) in a robotic drive. Cassette 220 has a distal end 222, a proximal end 224 and a longitudinal device axis 238 that is associated with and defined by an elongated medical device (EMD) 230 positioned in the cassette housing 228. In an embodiment, the EMD 230 is a catheter. The catheter 230 is coupled to a hemostasis valve (e.g., a rotating hemostasis valve (RHV)) 232 which is also positioned in the cassette housing 228. The hemostasis valve 232 includes a side port 234 that may be connected to a tube (not shown) to facilitate the flow of a fluid (e.g., saline) to and from the hemostasis valve 232 and the catheter 230. Cassette 220 also includes a cover 226 that is connected to the cassette housing 228 using a connection mechanism 236 (e.g., a hinge). The connection mechanism 236 is located at a position below the longitudinal device axis 238. In FIG. 11, the cover 226 is in a closed position. The connection mechanism 236 enables the cover 226 to be moved from the closed position to an open position.

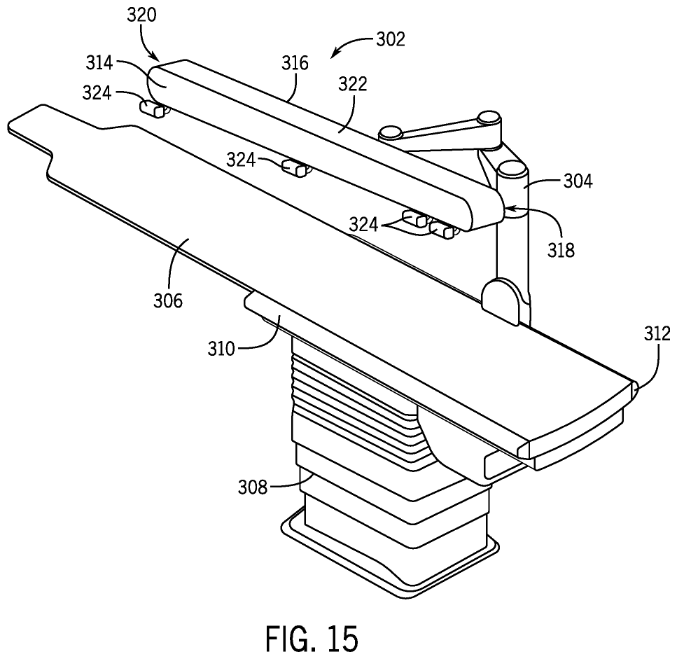

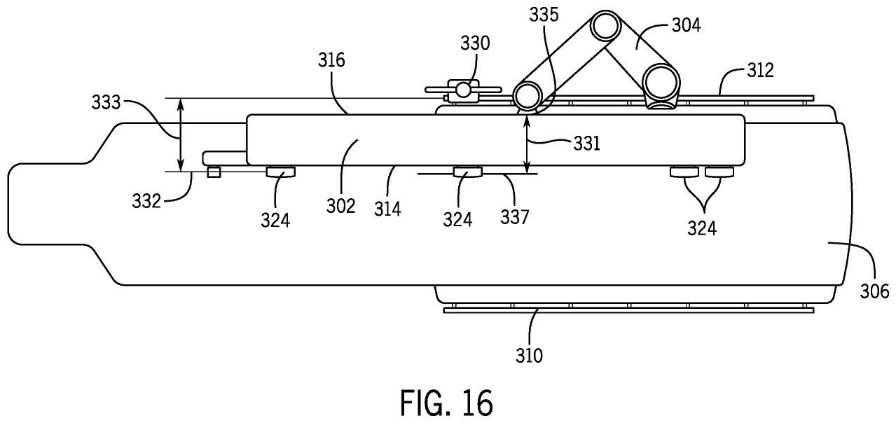

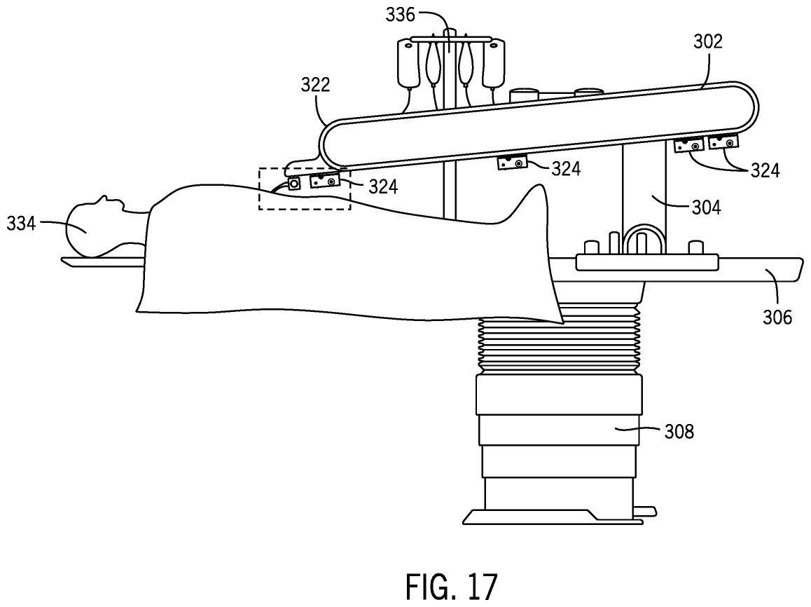

FIG. 15 is a perspective view of a bedside unit of a catheter-based procedure system mounted on a patient table in accordance with an embodiment. The bedside unit includes a robotic drive 302 (e.g., robotic drive 200 shown in FIG. 11a) and a positioning system 304. The robotic drive 302 has a housing 322 and four device modules 324 wherein each device module is configured to manipulate an EMD. In an embodiment, each device module 324 includes a vertically mounted drive module and a vertically mounted cassette. The robotic drive 302 has a front side 314, a rear (or back) side 316, a proximal end 318 and a distal end 320. The positioning system 304 (e.g., a robotic arm, an articulated arm, a holder, etc.) may be attached at one end to a patient table, for example, a rear rail 312 of the patient table 306. The other end of the positioning system 304 is attached to the robotic drive 302. The positioning system 304 may be used to situate or position the robotic drive 302 relative to a patient (not shown) on the patient table 306 for a procedure. The patient table 306 is operably supported by a pedestal 308 which is secured to the floor and/or earth. In an embodiment, the width of the robotic drive 302 limited to, for example, to avoid interference with other devices that may be mounted to the rear rail 312. FIG. 16 is a top view of a bedside unit of a catheter-based procedure system mounted on a patient table in accordance with an embodiment. In FIG. 16, a width 331 of the robotic drive is defined as a distance between an attachment or connection point 335 for the positioning system 304 to the robotic drive 302 and a longitudinal device axis 337 of the device modules 324. The longitudinal device axis 337 is associated with and defined by EMDs positioned in the device modules 324. In an embodiment, the width 331 is equal to or less than a distance 333 between an insertion point 332 where the introducer sheath would enter a femoral artery of a patient and the rear rail 312 of the patient table 306. In FIG. 16, the insertion point 332 is in a left femoral artery and the rear rail 312 of the patient table is a left rail. In other embodiments, the robotic drive 302, positioning system 304 and the patient (not shown) may be set up so that the insertion point is in the right femoral artery and the robotic device 302 and positioning system 304 are mounted to the front rail 310. In one embodiment, the width 331 of the robotic drive is approximately 15 cm. Having a width 331 as small as possible allows the robotic drive 302 to be parallel to other devices mounted on the rear rail 312 of the patient table 306 and still be able to have EMDs enter a femoral artery of a patient at the insertion point 332. By limiting the width 331 to be equal to or less than the distance 333 between the insertion point 332 and the rear rail 312, the robotic drive 302 may fit in the area between a groin (i.e., a femoral artery) of a patient and a rear rail 312 and will not run into or interfere with other devices on the rear rail 312 (e.g., IVs etc.), for example, a device mounted to a connection point 330 on the rear rail 312. FIG. 17 is a front view a bedside unit of a catheter-based procedure system mounted on a patient table in accordance with an embodiment. In FIG. 17, an example device mounted to a patient table 306 along with the positioning system 304 and the robotic drive 3o2 is an IV pole 336. The IV pole 336 is mounted to a rear rail (not shown) of the patient table 306.

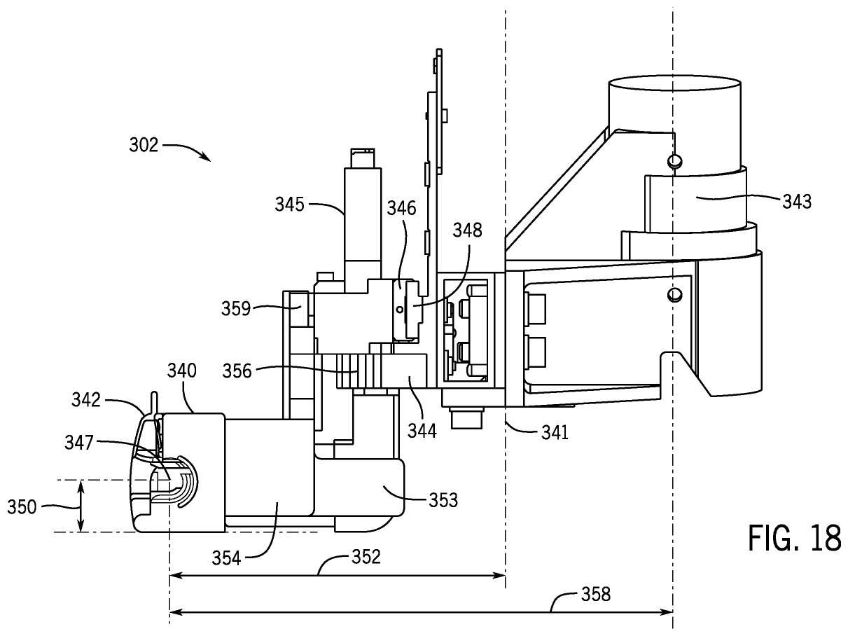

As mentioned, the robotic drive (200, 302) can be configured to minimize the width of the robotic drive and to allow the robotic drive to be placed close to the patient. FIG. 18 is a rear cross sectional view of a robotic drive with vertically mounted device modules in accordance with an embodiment. In FIG. 18, a cassette 340 is vertically mounted to a drive module 354. A cover 342 of the cassette 140 is shown in a closed position. The drive module 354 is coupled to a stage 346 which is movably coupled to a rail 348. In an embodiment, an offset bracket 359 may be used to couple the drive module 354 to the stage 346. The drive module 354 is also coupled with a motor 353 which is used to provide rotary motion to an EMD (not shown) positioned in the cassette 340 using, for example, a coupler as described above. A drive mechanism for the stage 346 includes a rack 344 and pinion 356. As described above, the pinion 356 may be directly connected to a motor 345 (e.g., a motor shaft) associated with the stage 346. The motor 345 is provided to drive the pinion 344 which is meshed with the rack 344 to provide liner motion to the stage 346. A width 352 of the robotic drive 302 is defined as a distance between an attachment point 341 for the positioning system 304 to the robotic drive 302 and a longitudinal device axis 347 of the device module (i.e., the cassette 340 and the drive module 354). The longitudinal device axis 347 is associated with and defined by an EMD (not shown) positioned in the cassette 340 of the device module. In an embodiment, the longitudinal device axis 347 is below or lower than a central axis of the cassette 340 to bring the longitudinal device axis closer to the patient. A distance 350 between the longitudinal device axis 347 and a bottom of the device module (e.g., the bottom of the cassette 340) can be reduced because the cassette 340 and drive module 354 are mounted vertically. Advantageously, in a vertical mount the drive module is not under the device axis and between the device axis and the patient. Accordingly, the longitudinal device axis 347 can get close to a patient, in particular, it is desirable to have the longitudinal device axis of the most distal device module (i.e., the device module closest to the patient along the linear member 211 (shown in FIG. 11a)) be as close to the patient as possible. In an embodiment, the cassette 340 is configured to minimize the distance 350. A distance 358 between the longitudinal device axis 347 and a positioning system interface 343 is also shown in FIG. 18. The positioning system interface 343 is coupled to, for example, the rear of the robotic drive and an arm of the positioning system. The positioning system interface 343 may be used to adjust a pitch angle of the robotic drive.

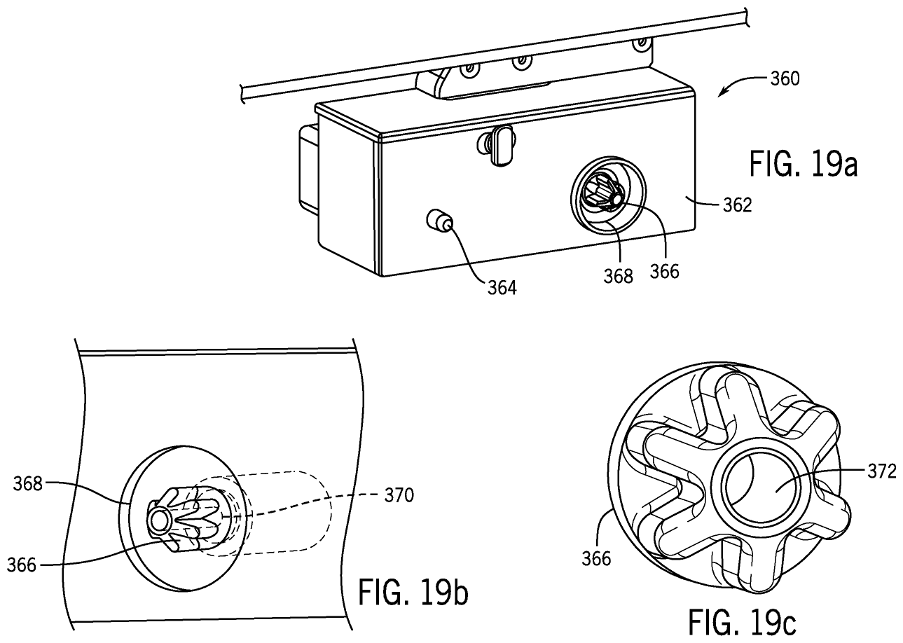

One element that can be configured to minimize the width of the robotic drive is the coupler of the drive module. FIG. 19a is a perspective view of a drive module in accordance with an embodiment, FIG. 19b is a front view of a motor shaft in a recess of a drive module in accordance with an embodiment and FIG. 19c is a perspective view of a coupler of a drive module in accordance with an embodiment. A drive module 360 includes a housing 362. A positioning pin 364 may be located on the front side of the drive module 360 (e.g., a mounting surface). A coupler 366 is positioned in a recess 368 of the housing 362. The positioning pin 364 can help to align the coupler 366 of the drive module 362 with a mating coupler, e.g., on a cassette (not shown), before the couplers are fully mated. The coupler 366 may be protected from external radial loads by positioning the coupler 366 in the recess 368 which can result in increased longevity of a motor bearing. As mentioned above, the coupler 366 may interface with a cassette (not shown) mounted on the drive module 360 and may be used to rotate an EMD in the cassette. For example, the cassette may include a bevel gear that interfaces with the coupler 366 of the drive module 360 and the bevel gear interfaces with a mating bevel gear which is coupled to an EMD in the cassette to rotate the EMD. To reduce the width of the robotic drive, the coupler 366 (e.g., a capstan) may be directly mounted to a shaft 370 of a motor in the drive module 360 as shown in FIG. 19b. In various embodiments, the capstan 366 may be directly mounted to the motor shaft 370 using, for example, a laser weld and/or adhesive or other permanent or non-permanent methods. In an embodiment, the coupled 366 (e.g., a capstan) may include an opening 372, shown in FIG. 19c, in which the motor shaft 370 may be inserted.

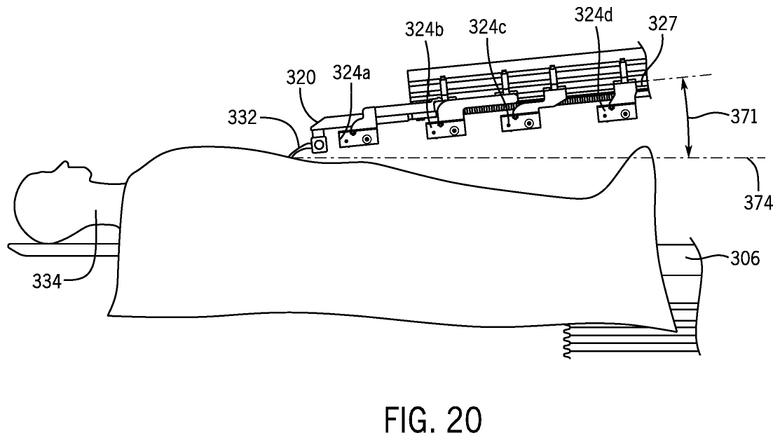

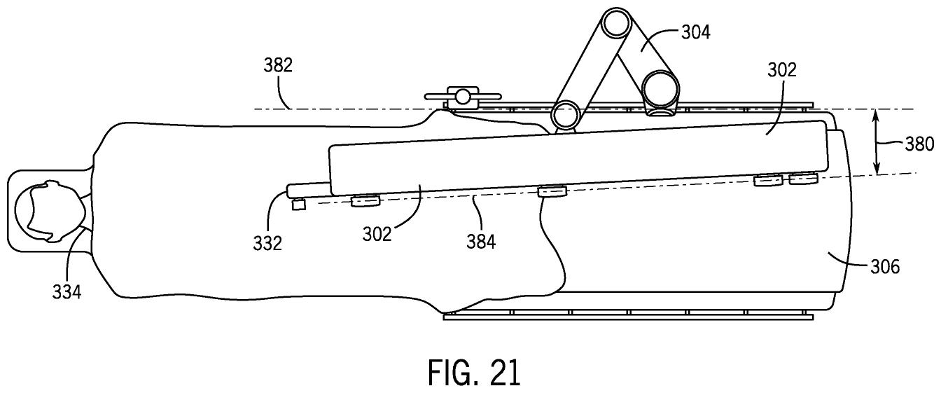

The angle at which the robotic drive (e.g., robotic drive 200 shown in FIG. 11a and robotic drive 302 shown in FIG. 15) is positioned can affect the usability of the robotic drive my making it difficult to load and unload EMDs due to the height of the more proximal device modules (e.g., device modules 324c, 324d), especially when the more proximal device modules are retracted to the proximal end of the robotic drive. In addition, the angle at which the robotic drive is positioned can make is difficult to adjust and handle the robotic drive. In addition, it can affect how close the most distal device module of the robotic drive can get to a patient. FIG. 20 is a front view of a robotic drive illustrating a pitch angle in accordance with an embodiment. In FIG. 20, a pitch angle 371 for a robotic drive 302 is defined between a horizontal axis 374 parallel to a patient table 306 and a proximal end of a rail 327. Device modules 324a-d are coupled to the stages 325a-d which are movably coupled to the rail 327. In an embodiment, the pitch angle 371 is less than 10 degrees. In another embodiment, the pitch angle 371 is on the range of 3-6 degrees. It is advantageous for the pitch angle 371 to be as small as possible for a long robotic drive system with multiple devices modules and EMDs. The pitch angle 371 of the robotic drive 302 should also be selected so that the robotic drive does not interfere with or come in contact with the patient's feet. Minimizing the pitch angle 371 can provides accessible loading height for devices. By limiting the width of the robotic drive as discussed above, the yaw of the robotic drive will not cause the robotic drive to interfere with other devices mounted on a rear rail of the patient table 306. FIG. 21 is a top view of a bedside unit of a catheter-based procedure system mounted on a patient table in accordance with an embodiment. In FIG. 21, the yaw is defined as the angle 380 between a longitudinal axis 382 of a rear rail of the patient table 306 and a longitudinal axis 384 of the robotic drive 302. It is desirable to minimize the yaw of the robotic drive 302 to avoid contact with other devices mounted on the rear rail of the patient table.

A control computing system as described herein may include a processor having a processing circuit. The processor may include a central purpose processor, application specific processors (ASICs), circuits containing one or more processing components, groups of distributed processing components, groups of distributed computers configured for processing, etc. configured to provide the functionality of module or subsystem components discussed herein. Memory units (e.g., memory device, storage device, etc.) are devices for storing data and/or computer code for completing and/or facilitating the various processes described in the present disclosure. Memory units may include volatile memory and/or non-volatile memory. Memory units may include database components, object code components, script components, and/or any other type of information structure for supporting the various activities described in the present disclosure. According to an exemplary embodiment, any distributed and/or local memory device of the past, present, or future may be utilized with the systems and methods of this disclosure. According to an exemplary embodiment, memory units are communicably connected to one or more associated processing circuit. This connection may be via a circuit or any other wired, wireless, or network connection and includes computer code for executing one or more processes described herein. A single memory unit may include a variety of individual memory devices, chips, disks, and/or other storage structures or systems. Module or subsystem components may be computer code (e.g., object code, program code, compiled code, script code, executable code, or any combination thereof) for conducting each module's respective functions.

This written description used examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. The order and sequence of any process or method steps may be varied or re-sequenced according to alternative embodiments.

Many other changes and modifications may be made to the present invention without departing from the spirit thereof. The scope of these and other changes will become apparent from the appended claims.

We claim:

1. A robotic drive system for a catheter-based procedure system, the robotic drive system comprising:
a positioning system coupled to a patient table, the patient table having a front side and a rear side, the rear side of the patient table having a rail;
a linear member coupled to the positioning system at a connection point; and
at least three device modules coupled to the linear member, each device module independently controllable and comprising:
a drive module having a front side; and
a cassette mounted on the drive module, the cassette having a front side and configured to support an elongated medical device having a longitudinal device axis, wherein the cassette is mounted on the drive module in a vertical orientation so that the front side of the cassette is parallel to the front side of the drive module;
wherein a width defined between the longitudinal device axis of the elongated medical device and the connection point of the linear member to the positioning system is equal to or less than a distance between an insertion point for the elongated medical device to a patient and the rail on the rear side of the patient table.

2. The robotic drive system according to claim 1, wherein the linear member comprises a drive mechanism to provide linear motion for each of the plurality of device modules and the drive mechanism is a rack and pinion linear actuator.

3. The robotic drive system according to claim 1, wherein the linear member comprises a drive mechanism to provide linear motion for each of the plurality of device modules and the drive mechanism is a screw.

4. The robotic drive system according to claim 2, wherein the linear member further comprises a rail positioned above the rack.

5. The robotic drive system according to claim 4, further comprising a plurality of stages moveably coupled to the rail wherein each stage in the plurality of stages is coupled to one of the plurality of device modules.

6. The robotic drive according to claim 5, wherein the rack and pinion linear actuator includes a plurality of pinions wherein each pinion is coupled to one of the stages in the plurality of stages and each stage includes a motor coupled to the pinion associated with the stage.

7. The robotic drive system according to claim 1, wherein each device module further comprises a bottom surface and a distance between the longitudinal device axis and the bottom surface of the device module is less than 20 mm.

8. The robotic drive system according to claim 1, wherein the insertion point for the elongated medical device is a femoral artery of the patient.

9. A robotic drive system for a catheter-based procedure system, the robotic drive system comprising:
a linear member; and
at least one device module coupled to the linear member, the at least one device module independently controllable and comprising:
a drive module comprising:
a housing having a front side including a recess;
a motor having a shaft, the motor disposed within the housing and the shaft positioned in the recess of the front side of the housing; and
a capstan directly mounted to the motor shaft; and
a cassette mounted on the drive module, the cassette having a front side and configured to support an elongated medical device having a longitudinal device axis, wherein the cassette is mounted on the drive module in a vertical orientation so that the front side of the cassette is parallel to the front side of the drive module and the cassette is coupled to the capstan.

10. The robotic drive system according to claim 9, wherein the capstan is directly mounted to the motor shaft using a laser weld.

11. The robotic drive system according to claim 9, further comprising a positioning system coupled to a patient table, the patient table having a front side and a rear side, the rear side of the patient table having a rail, wherein the linear member is coupled to the positioning system at a connection point and wherein a width defined between the longitudinal device axis of the elongated medical device and the connection point of the linear member to the positioning system is equal to or less than a distance between an insertion point for the elongated medical device to a patient and the rail on the rear side of the patient table.

12. The robotic drive system according to claim 9, wherein the linear member comprises a drive mechanism to provide linear motion for each of the plurality of device modules and the drive mechanism is a rack and pinion linear actuator.

13. The robotic drive system according to claim 9, wherein the linear member comprises a drive mechanism to provide linear motion for each of the plurality of device modules and the drive mechanism is a screw.

14. A robotic drive system for a catheter-based procedure system, the robotic drive system comprising:
a positioning system coupled to a patient table, the patient table having a front side and a rear side, the rear side of the patient table having a rail;
a linear member coupled to the positioning system at a connection point, the linear member having a distal end and a proximal end; and
at least three device modules coupled to the linear member, each device module independently controllable and configured to support an elongated medical device having a longitudinal device axis;
wherein the positioning system is configured to position the linear member and the at least three device modules at a pitch angle defined between a horizontal axis parallel to the patient table and the proximal end of the linear member, wherein the pitch angle is less than 10 degrees.

15. The robotic drive system according to claim 14, wherein each device module comprises:
a drive module having a front side; and
a cassette mounted on the drive module, the cassette having a front side and configured to support an elongated medical device having a longitudinal device axis, wherein the cassette is mounted on the drive module in a vertical orientation so that the front side of the cassette is parallel to the front side of the drive module.

16. The robotic drive system according to claim 14, wherein the linear member comprises a drive mechanism to provide linear motion for each of the plurality of device modules and the drive mechanism is a rack and pinion linear actuator.

17. The robotic system according to claim 14, wherein a width defined between the longitudinal device axis of the elongated medical device and the connection point of the linear member to the positioning system is equal to or less than a distance between an insertion point for the elongated medical device to a patient and the rail on the rear side of the patient table.

18. The robotic drive system according to claim 17, wherein the insertion point for the elongated medical device is a left femoral artery of the patient.

19. The robotic system according to claim 14, wherein the pitch angle is in the range of 3-6 degrees.
